(19) United States
(12) Patent Application Publication
(10) Pub. No.: US 2026/0137416 A1
(43) Pub. Date: May 21, 2026

(54) Tissue-Removing Catheter with Advancer Rail Assembly

(71) Applicant: Medtronic Vascular, Inc., Santa Rosa, CA (US)

(72) Inventors: Kevin Cunningham, Galway (IE); Cara MacGillivray, Gort (IE); Mark Murphy O'Brien, Ardnacrusha (IE); Tomas K. Kelly, Galway (IE); Clayton Vanderlinden, Menasha, WI (US); Scott Smieja, Oshkosh, WI (US)

(21) Appl. No.: 19/120,987

(22) PCT Filed: Oct. 12, 2023
(86) PCT No.: PCT/US2023/035022

§ 371 Date: Apr. 14, 2025

Related U.S. Application Data
(60) Provisional application No. 63/379,238, filed on Oct. 12, 2022.

(51) Int. Cl. A61B 17/3207; A61B 17/00; A61B 17/32

(52) U.S. Cl. CPC A61B 17/320758; A61B 2017/00398; A61B 2017/00778; A61B 2017/320004

(57) ABSTRACT

A tissue-removing catheter includes an elongate body, a handle, a motor, a tissue-removing element, and a slide assembly. The slide assembly is at least partially received in the handle and includes a carriage coupling a roller bearing and a track coupled to the handle. The carriage supports the motor. The track supports the roller bearing moving linearly along the track to enable the carriage and motor to selectively translate within the handle. At least one bearing may include a first rail bearing moveable within a first rail plane and a second rail bearing moveable within a second rail plane being angularly offset or generally transverse to the first rail plane. The track may include a rail having an interior surface defining an interior space extending along a length of the rail while at least one bearing engages the interior surface within the interior space.

### FIELD

The present disclosure generally relates to a tissue-removing catheter, and more particular, to a tissue-removing catheter having an advancer rail assembly.

### BACKGROUND

Tissue-removing catheters are used to remove unwanted tissue in body lumens. As an example, atherectomy catheters are used to remove material from a blood vessel to open the blood vessel and improve blood flow through the vessel. This process can be used to prepare lesions within a patient's coronary artery to facilitate percutaneous coronary angioplasty (PTCA) or stent delivery in patients with severely calcified coronary artery lesions. Atherectomy catheters typically employ a rotating element which is used to abrade or otherwise break up the unwanted tissue.

### SUMMARY OF DISCLOSURE

An aspect of the present disclosure provides a tissue-removing catheter for removing tissue in fca body lumen. The tissue-removing catheter includes an elongate body, a handle, a motor, a tissue-removing element, and a slide assembly. The elongate body is sized and shaped to be received in the body lumen and has an axis and proximal and distal end portions spaced apart from one another along the axis. The handle is at the proximal end portion of the elongate body. The motor is in the handle and is operatively coupled to the elongate body to drive rotation of the elongate body about the axis of the elongate body. The tissue-removing element is mounted on the distal end portion of the elongate body and removes the tissue as the tissue-removing element is rotated by the elongate body within the body lumen. The slide assembly is at least partially received in the handle and includes a carriage, at least one roller bearing coupled to the carriage, and a track. The carriage is coupled to and supports the motor. The track is coupled to the handle and supports the at least one roller bearing. The at least one roller bearing may move linearly along the track to enable the carriage and the motor to selectively translate within the handle.

In another aspect of the present disclosure, the at least one bearing may include a first rail bearing moveable within a first rail plane and a second rail bearing moveable within a second rail plane that is generally transverse to the first rail plane. The track is coupled to the handle and supports the at least one bearing of the slide assembly while the at least one bearing may move linearly along the track to enable the carriage and the motor to selectively translate within the handle. In the present aspect, the track includes a rail having an interior surface that defines an interior space extending along a length of the rail while at least one of the first rail bearing and the second rail bearing engages the interior surface within the interior space.

In yet another aspect of the present disclosure, the least one bearing is coupled to the carriage and may include a first rail bearing moveable within a first rail plane and a second rail bearing moveable within a second rail plane angularly offset to the first rail plane. The first track is coupled to the handle and supports the at least one bearing while the at least one bearing may move linearly along the first track to enable the carriage and the motor to selectively translate within the handle. The second track is coupled to the handle and supports the at least one bearing while the at least one bearing may move linearly along the first track to enable the carriage and the motor to selectively translate within the handle.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

FIG. 1 is a schematic illustration of a catheter of the present disclosure;

FIG. 2 is an enlarged elevation of a distal end portion of the catheter;

FIG. 3 is the enlarged elevation of a distal end portion of the catheter abrading though a lesion;

FIG. 4 is a cross section taken through line 4-4 in FIG. 2;

FIG. 5A is a top perspective of a handle of the catheter;

FIG. 5B is a top perspective of the handle with a top housing section removed;

FIG. 6 is a perspective of a gearbox housing and a motor coupled to a slide assembly and removed from the handle;

FIG. 7 is a perspective of a gear assembly in the handle;

FIG. 8 is a cross section view from line 8-8 in FIG. 5 of the handle housing a slide assembly;

FIG. 9 is a bottom view of the top housing section of the handle including the slide assembly;

FIG. 10 is a front view of the slide assembly;

FIG. 11 is a perspective view of the slide assembly;

FIG. 12 is a perspective view of the slide assembly in a middle housing section of the handle;

FIG. 13 is an alternative perspective view of the slide assembly;

FIG. 14 is a top view of a carriage of the slide assembly;

FIG. 15 is a perspective view of the carriage;

FIG. 16 is an alternative perspective view of the carriage;

FIG. 17 is an enlarged, end elevation view of the rail; and

FIG. 18 is similar to FIG. 17 including roller bearings received in the rail.

Corresponding reference characters indicate corresponding parts throughout the drawings.

### DETAILED DESCRIPTION

The present disclosure is generally directed to a tissue-removing catheter for removing tissue in a body lumen. In one embodiment, the catheter is an atherectomy device (e.g., rotational atherectomy device) suitable for removing (e.g., abrading, debulking, cutting, excising, ablating, etc.) occlusive tissue (e.g., embolic tissue, plaque tissue, atheroma, thrombolytic tissue, stenotic tissue, hyperplastic tissue, neoplastic tissue, etc.) from a vessel wall (e.g., coronary arterial wall, etc.). The catheter may be used to facilitate percutaneous coronary angioplasty (PTCA) or the subsequent delivery of a stent. Features of the disclosed embodiments may also be suitable for treating chronic total occlusion (CTO) of blood vessels and stenosis of other body lumens and other hyperplastic and neoplastic conditions in other body lumens, such as the ureter, the biliary duct, respiratory passages, the pancreatic duct, the lymphatic duct and the like. Neoplastic cell growth will often occur as a result of a tumor surrounding and intruding into a body lumen. Removal of such material can thus be beneficial to maintain patency of the body lumen.

The following is a description of a suitable tissue-removing catheter in which embodiments of the present disclosure may be incorporated. It is understood that this description of a suitable tissue-removing catheter is non-limiting, and a suitable tissue-removing catheter may omit certain component and/or include additional component(s).

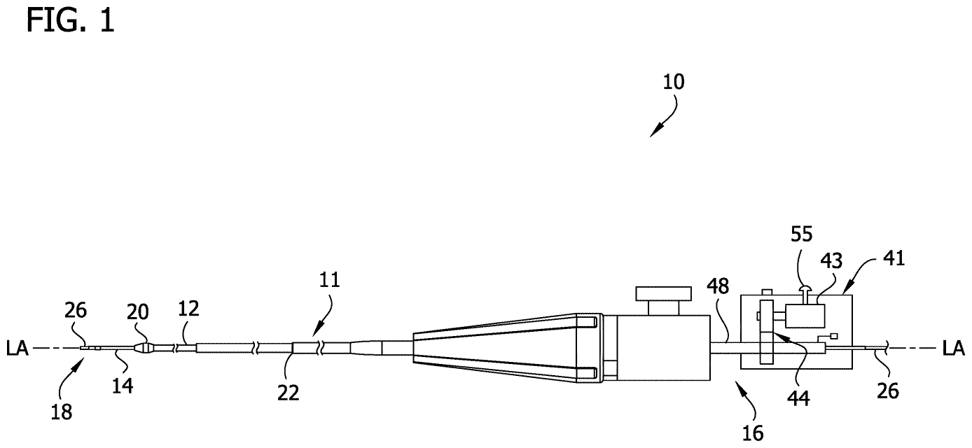

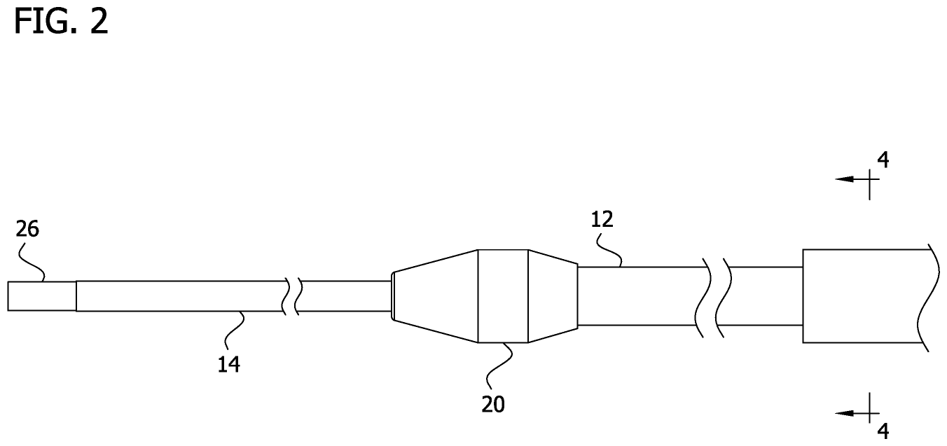

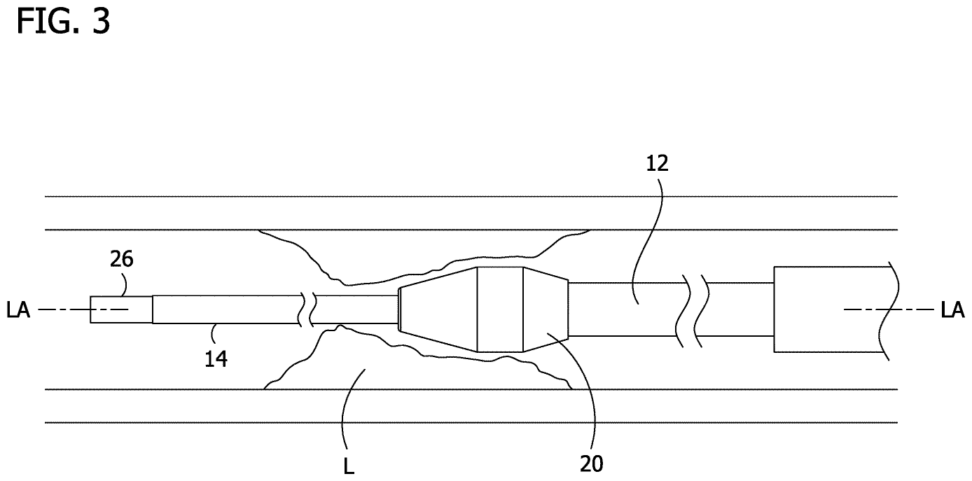

Referring to FIGS. 1-3, an exemplary embodiment of a rotational tissue-removing catheter for removing tissue (e.g., a lesion L) in a body lumen is generally indicated at reference number 10. The illustrated catheter 10 includes an elongate catheter body, generally indicated at reference numeral 11 (FIG. 1), having proximal and distal end portions. In one example, the catheter body 11 is sized for being received in a blood vessel of a subject. Thus, the catheter body 11 may have a maximum size of 3, 4, 5, 6, 7, 8, 9, 10, or 12 French (1, 1.3, 1.7, 2, 2.3, 2.7, 3, 3.3, or 4 mm) and may have a working length of 20, 30, 40, 60, 80, 100, 120, 150, 180 or 210 cm depending on the body lumen. While the remaining discussion is directed toward a catheter for removing tissue in blood vessels, it will be appreciated that the teachings of the present disclosure also apply to other types of tissue-removing catheters, including, but not limited to, catheters for penetrating and/or removing tissue from a variety of occlusive, stenotic, or hyperplastic material in a variety of body lumens.

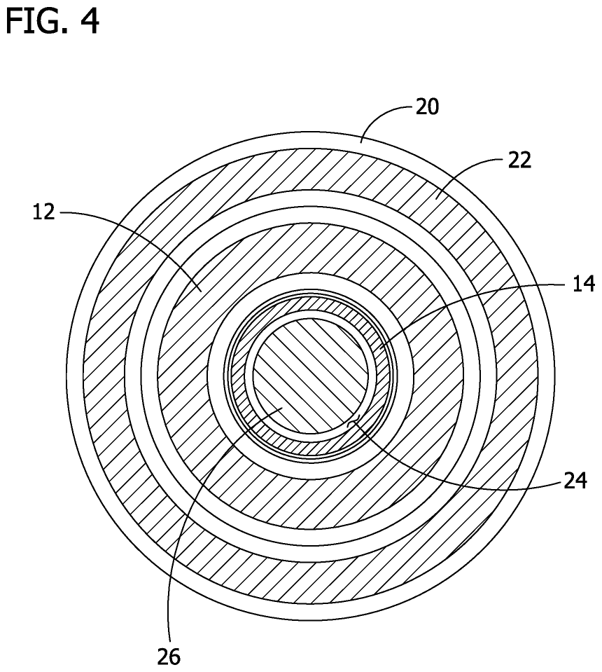

Referring to FIGS. 1-4, the catheter body 11 comprises an elongate drive shaft or drive coil 12 (broadly, an elongate body) disposed around an elongate inner liner 14. The drive coil 12 and inner liner 14 extend along a longitudinal axis LA of the catheter from a proximal end portion 16 to a distal end portion 18 of the catheter. A tissue-removing element 20 (e.g. an abrasive burr) is disposed on a distal end portion of the catheter body 11 (e.g., on the drive coil 12). The tissue-removing element 20 is configured for rotation by rotation of the catheter body to remove tissue, such as a lesion L, from a body lumen. The abrasive burr 20 may have an abrasive outer surface formed, for example, by a diamond grit coating, surface etching, or the like. A sheath 22 (FIG. 1) is disposed around the drive coil 12. The drive coil 12 and the inner liner 14 are both configured to translate relative to the sheath 22, as described below. The catheter 10 is sized and shaped for insertion into a body lumen of a subject. The sheath 22 isolates the body lumen from at least a portion of the drive coil 12 and inner liner 14. The inner liner 14 defines a guidewire lumen 24 for slidably receiving a guidewire 26 therein so that the catheter 10 can be advanced through the body lumen by traveling along the guidewire. The guidewire can be a standard 0.014-inch outer diameter, 300 cm length guidewire. In certain embodiments, the inner liner 14 may have a lubricious inner surface for sliding over the guidewire 26 (e.g., a lubricious surface may be provided by a lubricious polymer layer or a lubricious coating). In the illustrated embodiment, the guidewire lumen 24 extends from the proximal end portion 16 through the distal end portion 18 of the catheter 10 such that the guidewire 26 is extendable along an entire working length of the catheter 10. In one embodiment, the overall working length of the catheter 10 may be between about 130 cm (51.2 inches) and about 160 cm (63 inches). In use, the guidewire 26 may extend about 40 mm (1.6 inches) past a distal end of the inner liner 14.

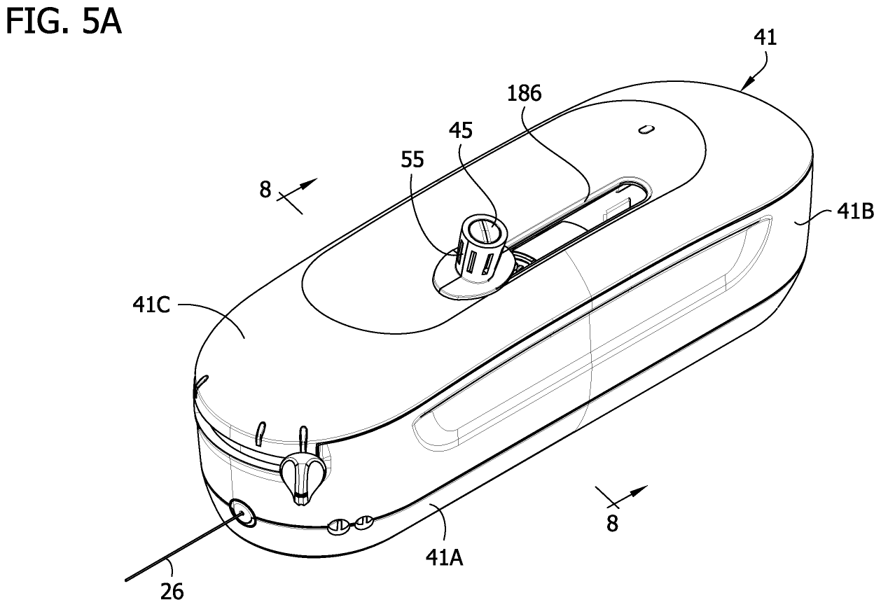

Referring to FIGS. 5A and 5B, the catheter 10 further comprises a handle, generally indicated at 41, coupled to a proximal end of the catheter body 11, such as at the isolation sheath 22. The handle 41 is configured to support components therein. In the illustrated embodiment, the handle 41 includes a bottom housing section 41A, a middle housing section 41B secured to the top of the bottom housing section, and a top housing section 41C secured to the top of the middle housing section. It will be understood that the handle 41 can have other shapes and configurations without departing from the scope of the disclosure.

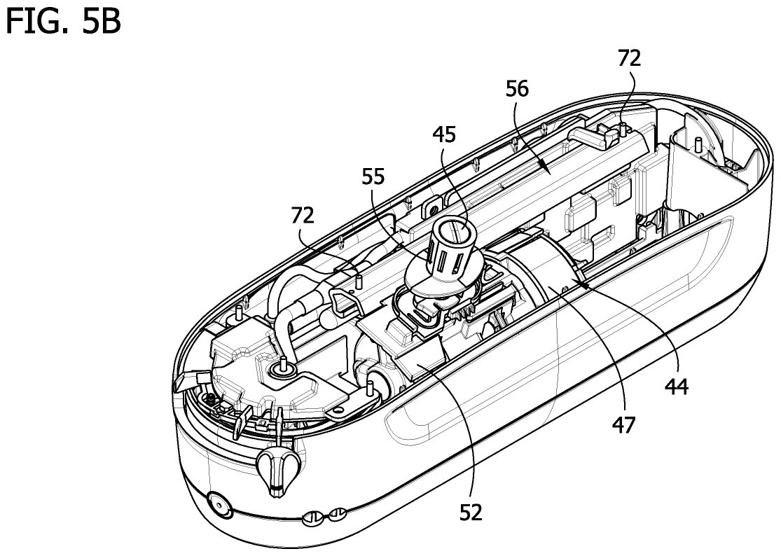

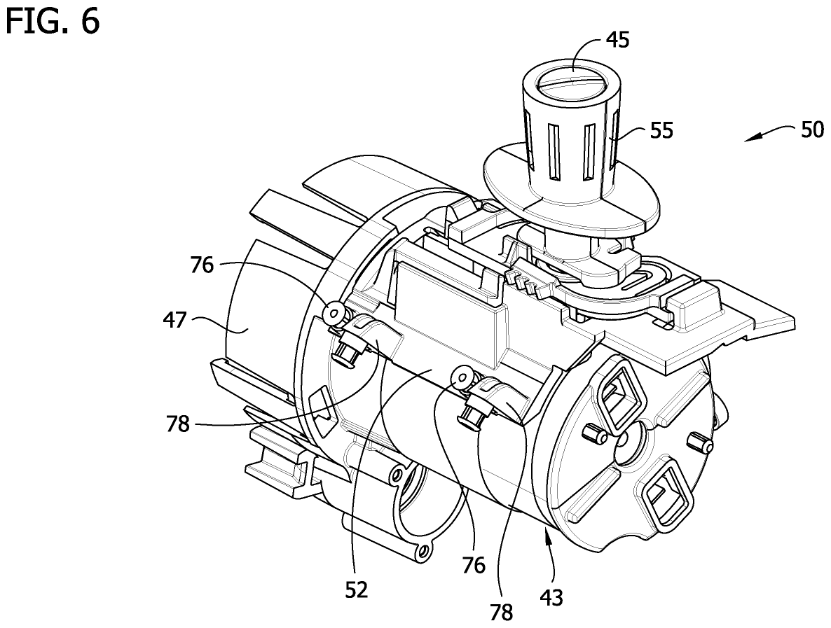

In the illustrated embodiment, the handle 41 supports a motor actuator 45 (e.g., a lever, a button, a dial, a switch, or other device) configured for selectively actuating a motor 43 (e.g., an electric motor), disposed in the handle to drive rotation of the elongate body (e.g., the drive coil 12) and the tissue-removing element 20 mounted at the distal end of the elongate body. The motor 43 is operatively coupled to the drive coil 12 and configured to rotate the drive coil 12 and the tissue-removing element 20 at speeds of up to, for example but not limiting to, about 100,000 RPM. In the illustrated embodiment, the motor 43 is coupled to the drive coil 12 by a gear assembly 44 and drive assembly 48 (broadly, a motor drivetrain output) supported within the handle 41. Referring to FIGS. 6 and 7, the gear assembly 44 comprises a gearbox housing 47 (FIGS. 5B and 6) that mounts and at least partially encloses a pair of gears 81, 83 (FIG. 7) for transferring the rotation of a shaft of the motor 43 to the drive coil 12. In particular, a driven gear 83 is in mesh with the driver gear 81 so that rotation of the driver gear causes the driven gear to rotate in the opposite direction. Thus, when current is applied to the motor 43 from a power source, the electrical energy from the current is converted into mechanical energy to rotate the gear assembly 44 for rotating the drive coil 12. It is understood that the motor 43 may be coupled to the elongate body (e.g., drive coil 12) in other ways for transferring rotation (torque) from the motor to the elongate body.

Referring to FIGS. 1, 6 and 8-10, the tissue-removing catheter 10 includes a slide assembly, generally indicated at 50, at least partially received in the handle 41. The slide assembly 50 includes a carriage 52 coupled to and supporting at least the motor 43 (a motor shroud of the motor being visible in FIG. 6); at least one bearing (e.g., first, second and third roller bearings 54A, 54B, 54C) coupled to the carriage (the roller bearings being omitted in FIG. 6); a slide actuator 55 coupled to the carriage to enable a user to operate the slide assembly; and at least one track, generally indicated at 56, coupled to the handle. The slide assembly 50 enables a user to linearly advance the motor 43 (and the gear assembly 44 in one embodiment) within the handle 41 to impart linear movement to the drive coil 12 and the tissue-removing element 20 relative to the handle. For example, the slide assembly 50 may allow from about 30 mm to about 70 mm or more of linear advancement of the tissue-removing element 20. In the illustrate embodiment, the slide actuator 55 comprises a post, knob, or other structure to enable a user's hand to manually operate the slide assembly 50. The motor actuator 45 is coupled to the slide actuator 55 in the illustrated embodiment. In one or more other embodiments, the motor actuator may be located elsewhere on the handle or separate from the handle.

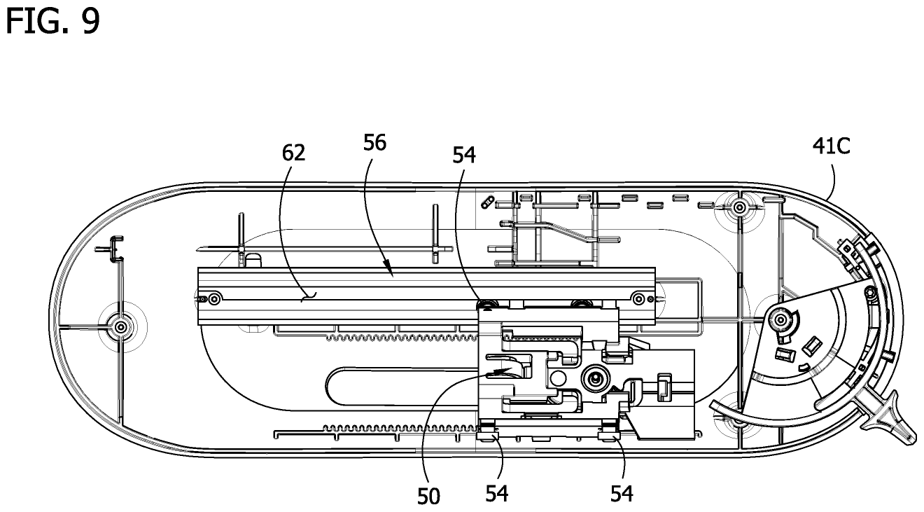

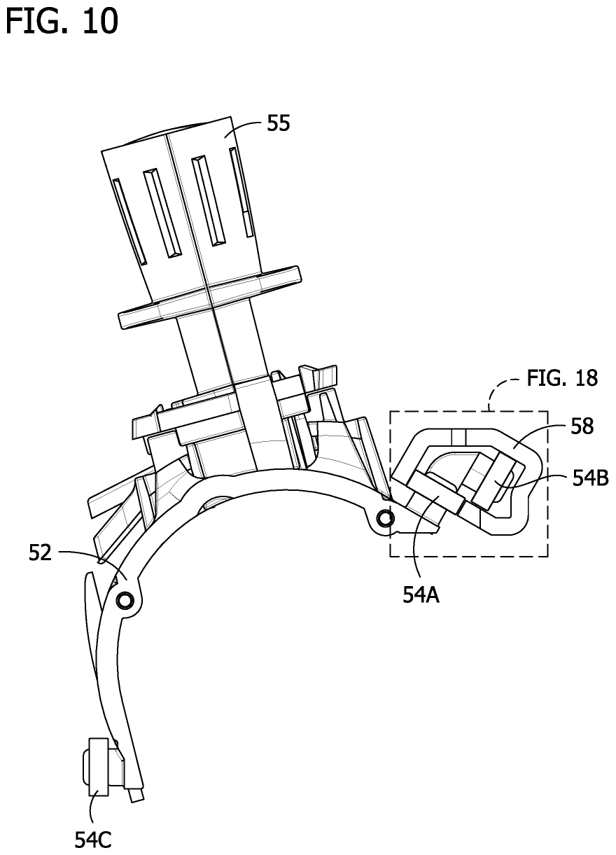

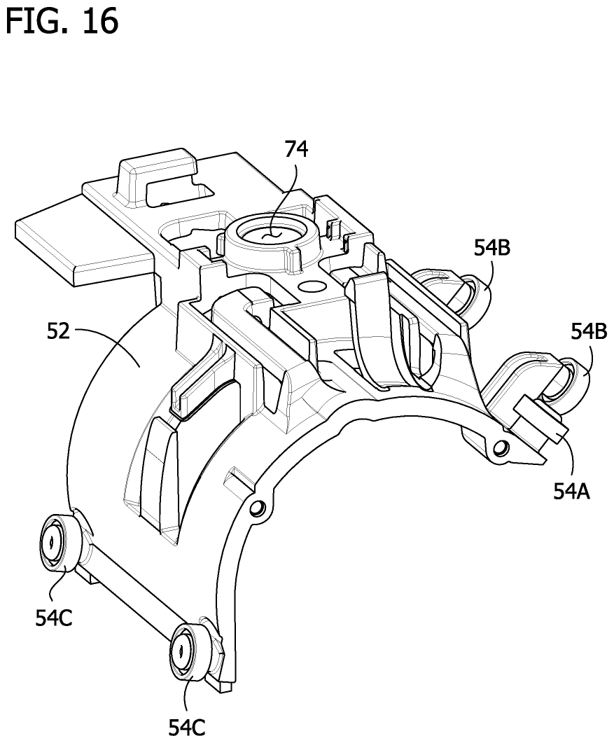

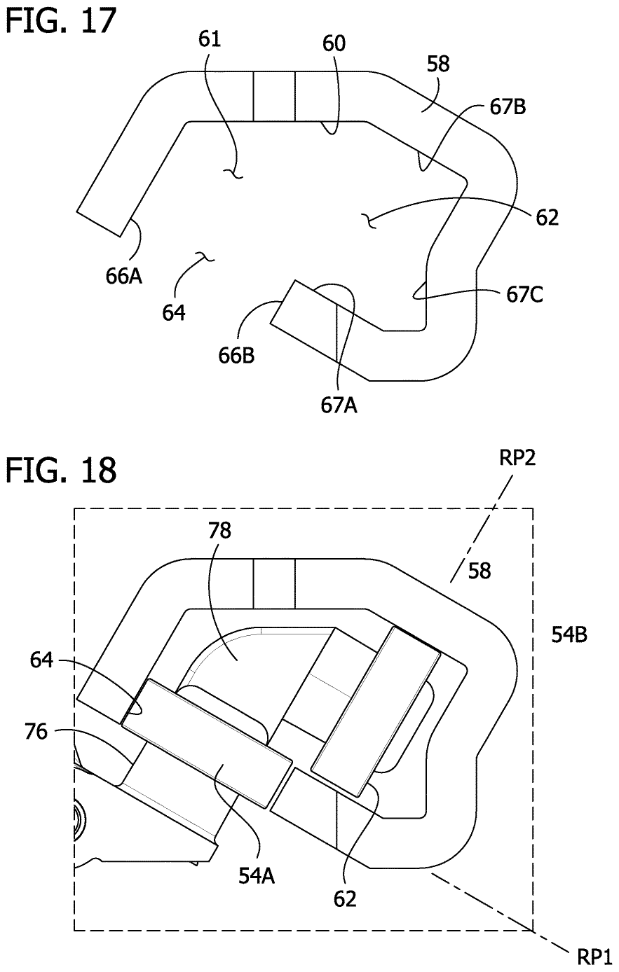

As shown in FIGS. 17 and 18, the track 56 of the slide assembly 50 includes a rail 58 having an interior surface 60 that defines an interior space 61 including an elongate internal channel 62 extending along a length of the rail. The rail 58 further defines a slot 64 generally between opposing first and second interior surface portions 66A, 66B of the rail. As shown in FIGS. 10 and 11, the channel 62 and slot 64 extend along the length of the rail 58, and the slot 64 opens to the channel. At least one of the roller bearings (e.g., the first and second roller bearings 54A and 54B) is coupled to and moves linearly along the track 56 to enable the carriage 52 and the motor 43 to selectively translate within the handle 41. In the illustrated embodiment, a plurality of first roller bearings 54A (e.g., two first roller bearings) is received in the slot 64 of the interior space 61 and configured ride along (i.e., rotatably engage) the first and second surface portions 66A, 66B of the rail 58. Moreover, a plurality of second roller bearings 54B (e.g., two second roller bearings) is received in the channel 62 and configured to ride along (i.e., rotatably engage) opposing third and fourth interior surface portions 67A, 67B of the rail 58. As shown in FIG. 10, the third and fourth interior surfaces 67A, 67B of the rail 58 are planar connected by a cross-wise interior surface portion 67C extending generally perpendicular to the surface portions 67A, 67B to aid in maintaining position of the roller bearings 54B in the channel 62. The illustrated side surface portion 67C has a convex (e.g., V-shape to restrict lateral movement (e.g., rattle) of the roller bearings 54B in the channel 62 during translation of the carriage 52 along the rail 58.

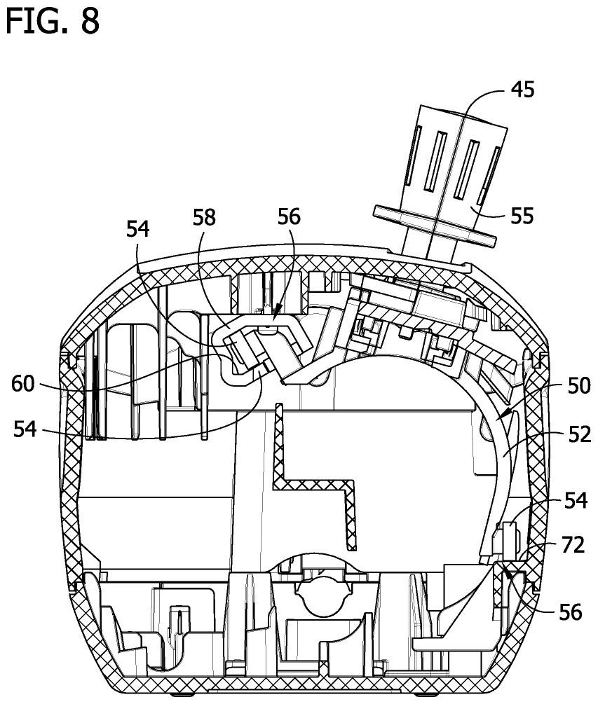

As shown in FIG. 9, posts or studs in the handle 41 (e.g., extending from the upper housing section 41C) are received and secured in openings extending through the rail 58 to couple the rail to the handle 41. The studs in the handle 41 and the openings in the rail 58 are spaced apart along a length such that the rail is secured to the handle by the studs through the openings at end portions of the rail. In an embodiment, when the studs are received in the openings in the rail 58, fasteners (e.g., screws) secure the rail to the handle 41. In other embodiments, alternative fastening methods may be used without departing from the present disclosure such that rail 58 may be coupled to the housing 41 in other ways.

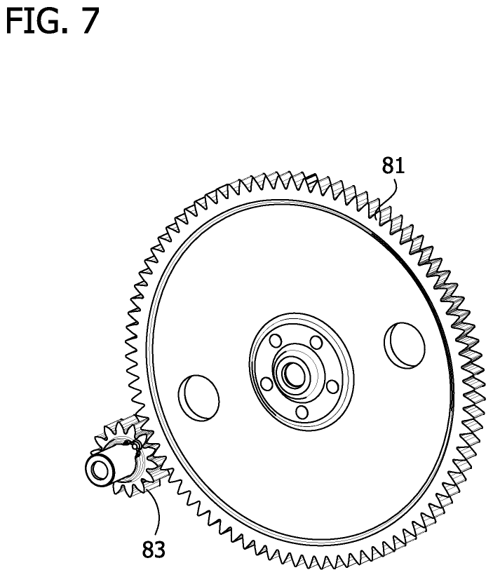

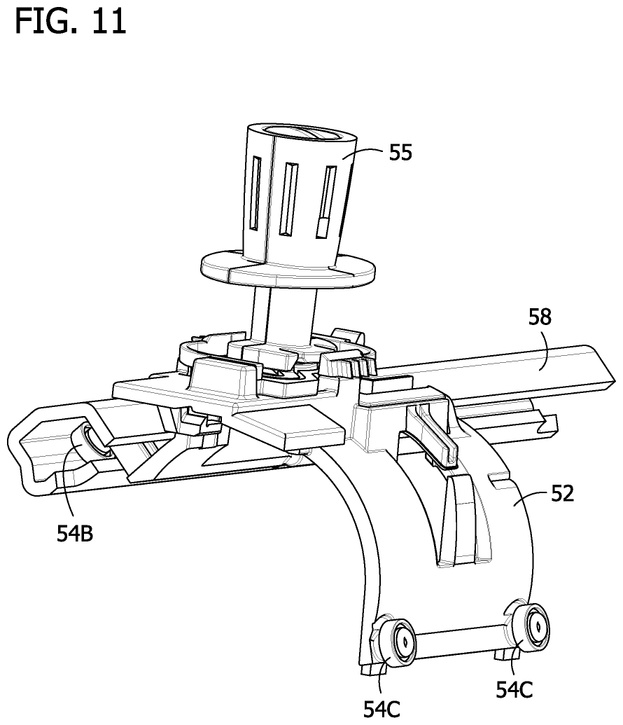

In the illustrated embodiment, the track 56 further includes an internal shoulder 72, as shown in FIGS. 8 and 12, spaced apart from and generally parallel to a portion of the rail 58 (e.g., the top surface of the rail) within the handle 41. At least one third roller bearing 54C engages the internal shoulder 72. The shoulder 72 may be integrally formed with or otherwise connected to the housing 41 (e.g., the middle housing section 41B). In this embodiment, both the rail roller bearings 54A, 54B and the shoulder bearings 54C aid in longitudinally translating and supporting the carriage 52 in the handle 41, and inhibiting the carriage from shifting laterally within the handle.

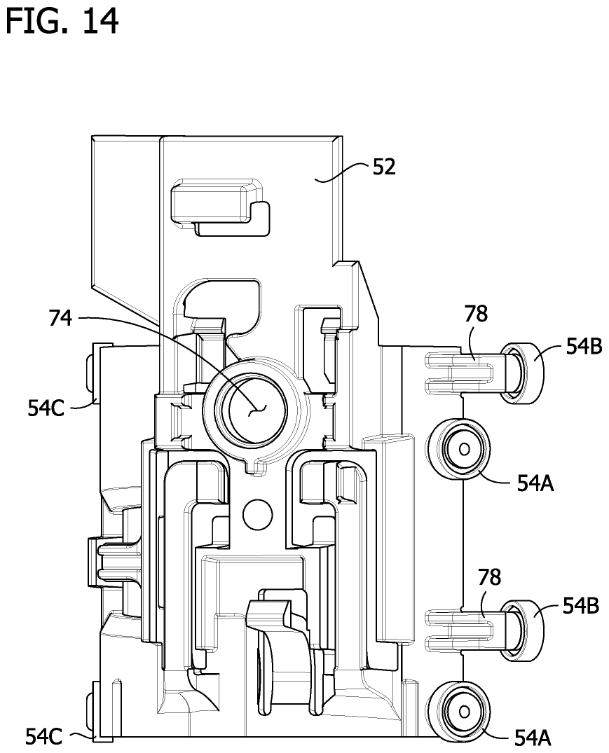

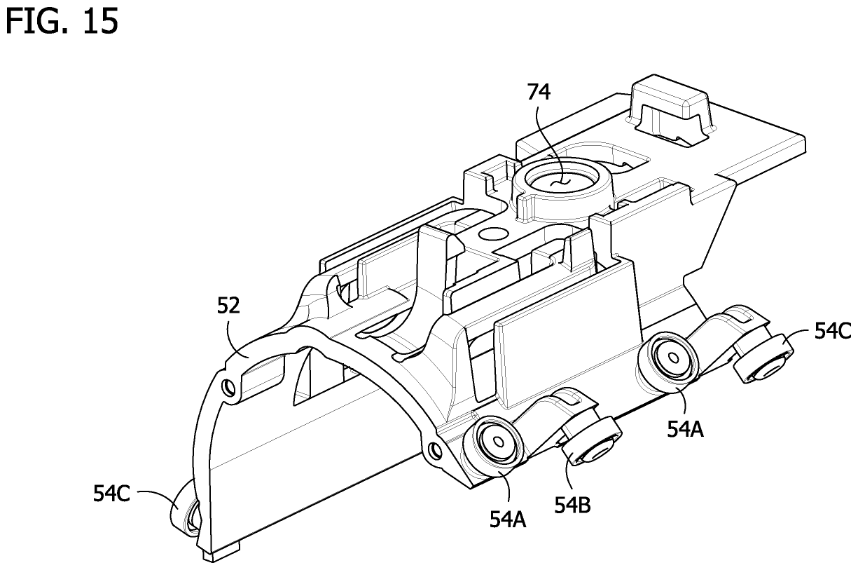

Referring to FIGS. 10-16, at least a portion of the carriage 52 has a generally arcuate cross-sectional shape having opposite first and a second arc end portions. An inner surface of the arcuate portion of the carriage 52 is configured to cradle the motor 43 (FIGS. 1 and 6). The roller bearings may be oriented on the carriage 52 such that the first and second roller bearings 54A, 54B (e.g., rail roller bearings) are coupled to the first arc end portion and the third roller bearing(s) (i.e., shoulder bearing(s)) is coupled to the second arc end portion. The illustrated first and third roller bearings 54A, 54C are coupled to the carriage 52 via stems or posts 76, 77, respectively (e.g., a straight stems). The second roller bearings 54B are coupled to the carriage 52 via stems or posts 78 (e.g., angled stems). The carriage and the roller bearings may have other configurations. In an embodiment, heat staking is used to melt and mold a portion of the roller bearings 54A, 54B, 54C to their respective stems or posts 76, 77, 78, securing the roller bearings on the stems or posts while allowing the roller bearings to rotate such that the carriage 52 moves linearly along the track 56. In other embodiments, alternative ways of securing the roller bearings 54A, 54B, 54C on their respective stems or posts 76, 77, 78 may be used as an alternative to heat staking.

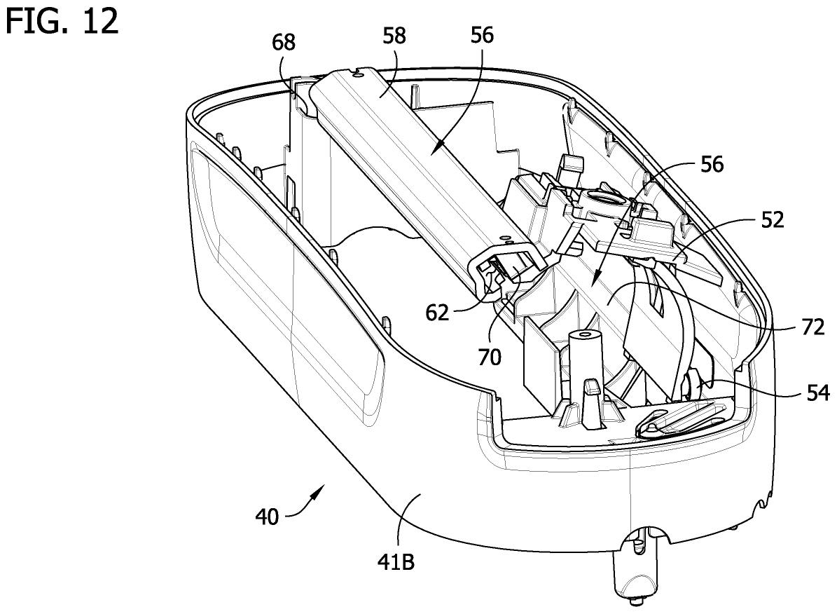

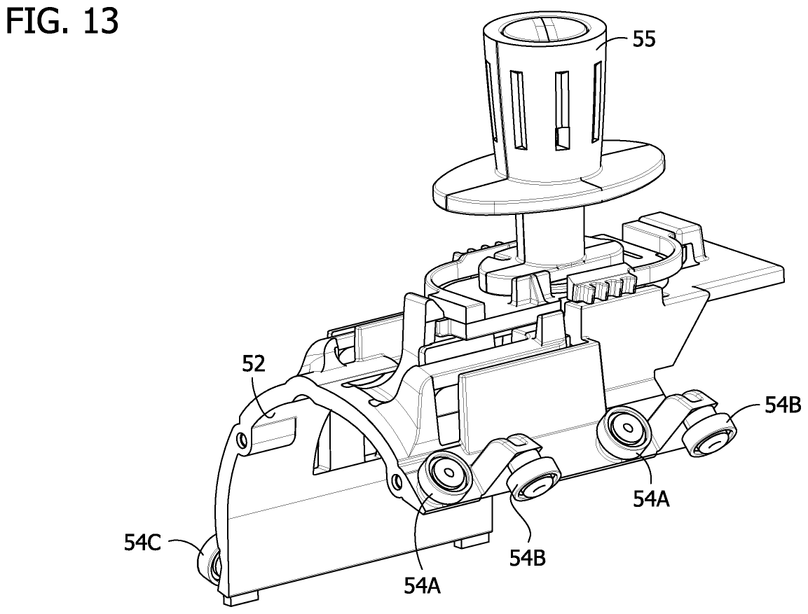

As shown in FIG. 18, the first roller bearings 54A (i.e., the first rail roller bearings) are rotatable within a first rail roller plane RP1, and the second roller bearings 54B (i.e., the second rail roller bearings) are rotatable within a second rail roller plane RP2. The first and second rail roller planes of the rail roller bearings 54A, 54B are generally transverse (e.g., orthogonal) to one another. Moreover, the second roller bearings 54B are laterally offset from the first roller bearings 54A. Orientation of the first and second rail roller bearings 54A, 54B are configured to secure the carriage 52 on the rail 58 while minimizing catch of the carriage and play between the carriage and the rail when translating along the rail. In other words, the configuration of the first and second roller bearings 54A, 54B restricts lateral movement of the carriage 52, the gearbox 44, and the motor 43 in two planes (e.g., X and Y planes) as the slide assembly 50 is advanced linearly along the handle 41 (e.g., along a Z-plane). This restriction in two planes better supports the motor 43 and gearbox 44 as compared to restricting movement in a single plane. This configuration also aids in better stabilizing the motor 43 and the gearbox 44 relative to the handle 41, as compared to a single plane restriction, to inhibit or at least reduce lateral movement or shaking of the motor 43 and gearbox 44 during operation of the motor. As shown in FIGS. 13-16 as described above, the slide assembly 50 includes a plurality of first and second rail roller bearings 54A, 54B. As seen best in FIG. 14, the first rail roller bearings 54A are spaced apart from one another along the length of the rail 58, the second rail roller bearings 54B are spaced apart from one another along the length of the rail, and the first and second roller bearings are spaced apart from each other to have a plurality of contact points (e.g., a current location of a bearing on the track 56) between the first and second rail roller bearings and the rail simultaneously. It is believed that having two or more of each roller bearing 54A, 54B provides additional support for the motor 43 and the gearbox 44, although in some embodiments, one roller bearing of each may be suitable. In an embodiment, one of the plurality of the second rail roller bearings 54B is disposed longitudinally between adjacent ones of the plurality of first rail roller bearings 54A, and one of the plurality of first rail roller bearings is between adjacent ones of the plurality of second rail roller bearings. The rail roller bearings 54A, 54B may have other orientations and/or configurations.

As shown in FIGS. 13-16, the third roller bearings 54C (i.e., the shoulder bearings) are configured to roll along the internal shoulder 72 of the track 56 in a plane generally transverse (e.g., orthogonal) to a portion of the internal shoulder to linearly advance the carriage. As shown in FIG. 12, the internal shoulder 72 of the track 56 extends inward relative to the housing 41 (e.g., middle housing section 41B) forming a top surface. The shoulder bearings 54C sit on the top surface of the internal shoulder 72 of the track 56. When the carriage 52 translates along the track 56, the shoulder bearing 54C rolls along the top surface of the internal shoulder 72 such that the carriage moves linearly. The interaction of the third roller bearings 54C with the shoulder 72 provides additional support for the carriage 52 so that both arc ends of the carriage are supported within the housing 41. The third roller bearings 54C may have other configurations and, in one or more embodiments, may be omitted from the handle 41.

As shown in FIGS. 14-16, the carriage 52 includes an opening 74 for coupling the slide actuator 55 to the carriage, such as by a coupling mechanism such as a press fit nut in the opening of the carriage with the press fit nut having internal threads for fastening the slide actuator to the carriage. In other embodiments, alternative coupling mechanisms may be used without departing from the scope of the disclosure. As shown in FIG. 10, the slide actuator 55 is radially offset from the longitudinal axis LA (FIG. 1) of the catheter 10 for one-handed control of the catheter and to allow the slide assembly to be operable without interfering with other components within the handle 41. As shown in FIG. 5, a portion of the handle 41 (e.g., top housing section) defines a slot 86 which limits the movement of the slide actuator 55 relative to the handle. Thus, the length of the slot 86 of the handle 41 determines the amount of relative movement of the carriage 52 within the handle 41, and thereby between the drive coil 12 and the handle.

The invention may be further described by reference to the following numbered paragraphs:
1. A tissue-removing catheter for removing tissue in a body lumen, tissue-removing catheter comprising:
   an elongate body having an axis and proximal and distal end portions spaced apart from one another along the axis, the elongate body being sized and shaped to be received in the body lumen;
   a handle at the proximal end portion of the elongate body;
   a motor in the handle, the motor being operatively coupled to the elongate body to drive rotation of the elongate body about the axis of the elongate body;
   a tissue-removing element mounted on the distal end portion of the elongate body, the tissue-removing element being configured to remove the tissue as the tissue-removing element is rotated by the elongate body within the body lumen; and
   a slide assembly at least partially received in the handle, the slide assembly including
      a carriage coupled to and supporting the motor,
      at least one roller bearing coupled to the carriage, and
      a track coupled to the handle and supporting the at least one roller bearing,
   wherein the at least one roller bearing is configured to move linearly along the track to enable the carriage and the motor to selectively translate within the handle.
2. The tissue-removing catheter according to paragraph 1, wherein the track includes a rail having an interior surface defining an interior space extending along a length of the rail, wherein the at least one roller bearing includes at least one rail roller bearing disposed within the interior space.
3. The tissue-removing catheter according to paragraph 2, wherein the interior surface of the rail includes opposing surface portions and a cross-wise surface portion extending between the opposing surface portions together defining a channel of the interior space, wherein the at least one rail roller bearing is captured within the channel of the interior space.
4. The tissue-removing catheter according to paragraph 3, wherein the at least one rail roller bearing rotatably engages the opposing surface portions of the interior surface of the rail defining the channel.
5. The tissue-removing catheter according to paragraph 2, wherein the at least one rail roller bearing includes at least one first rail roller bearing rotatable within a first rail roller plane, and at least one second rail roller bearing rotatable within a second rail roller plane that is generally transverse to the first rail roller plane.
6. The tissue-removing catheter according to paragraph 5, wherein the at least one first rail roller includes a plurality of first rail roller bearings spaced apart from one another along length of the carriage.
7. The tissue-removing catheter according to paragraph 6, wherein the at least one second rail roller bearing includes a plurality of second rail roller bearings spaced apart from one another along the length of the carriage.
8. The tissue-removing catheter according to paragraph 6, wherein the at least one second rail roller bearing is disposed longitudinally between adjacent ones of the plurality of first rail roller bearings relative to the length of the carriage.
9. The tissue-removing catheter according to paragraph 2, wherein the interior surface of the rail includes opposing surface portions extending between the opposing surface portions together defining a slot, wherein the at least one rail roller bearing is disposed within the slot.
10. The tissue-removing catheter according to paragraph 2, wherein the track further includes an internal shoulder spaced apart from and generally parallel to the length of the rail, wherein the at least one roller bearing further includes at least one shoulder roller bearing rotatably engaging the internal shoulder.
11. The tissue-removing catheter according to paragraph 1, wherein the track includes an internal shoulder extending inward relative to the handle.
12. A tissue-removing catheter for removing tissue in a body lumen, tissue-removing catheter comprising:
   an elongate body having an axis and proximal and distal end portions spaced apart from one another along the axis, the elongate body being sized and shaped to be received in the body lumen;
   a handle at the proximal end portion of the elongate body;
   a motor in the handle, the motor being operatively coupled to the elongate body to drive rotation of the elongate body about the axis of the elongate body;
   a tissue-removing element mounted on the distal end portion of the elongate body, the tissue-removing element being configured to remove the tissue as the tissue-removing element is rotated by the elongate body within the body lumen; and
   a slide assembly in the handle, the slide assembly including
      a carriage coupled to and supporting the motor,
      a first rail bearing coupled to the carriage and moveable within a first rail plane,
      a second rail bearing coupled to the carriage and moveable within a second rail plane that is generally transverse to the first rail plane, and
      a rail coupled to the handle and supporting the first and second rail bearings,
   wherein the first and second rail bearings are configured to move linearly along the rail to enable the carriage and the motor to selectively translate within the handle, the rail having an interior surface defining an interior space extending along a length of the rail,
   wherein at least one of the first rail bearing and the second rail bearing is disposed within the interior space.
13. The tissue-removing catheter according to paragraph 12, wherein the first rail bearing includes a plurality of first rail bearings spaced apart from one another along a length of the carriage.
14. The tissue-removing catheter according to paragraph 13, wherein the second rail bearing includes a plurality of second rail bearings spaced apart from one another along the length of the carriage.
15. The tissue-removing catheter according to paragraph 13, wherein the second rail bearing is disposed longitudinally between adjacent ones of the plurality of first rail bearings relative to the length of the carriage.
16. The tissue-removing catheter according to paragraph 12, wherein the second rail plane is generally orthogonal to the first rail plane.
17. The tissue-removing catheter according to paragraph 12, wherein the slide assembly further includes an internal shoulder spaced apart from and generally parallel to the length of the rail, and a shoulder bearing coupled to the carriage and engaging the internal shoulder, wherein the shoulder bearing is configured to move linearly along the internal shoulder to enable the carriage and the motor to selectively translate within the handle.
18. A tissue-removing catheter for removing tissue in a body lumen, tissue-removing catheter comprising:
   an elongate body having an axis and proximal and distal end portions spaced apart from one another along the axis, the elongate body being sized and shaped to be received in the body lumen;
   a handle at the proximal end portion of the elongate body;
   a motor in the handle, the motor being operatively coupled to the elongate body to drive rotation of the elongate body about the axis of the elongate body;
   a tissue-removing element mounted on the distal end portion of the elongate body, the tissue-removing element being configured to remove the tissue as the tissue-removing element is rotated by the elongate body within the body lumen; and
   a slide assembly in the handle, the slide assembly including
      a carriage coupled to and supporting the motor,
      a first bearing coupled to the carriage and moveable within a first plane,
      a second bearing coupled to the carriage moveable within a second plane angularly offset to the first plane,
      a first support surface coupled to the handle and supporting the first bearing,
   wherein the first bearing is configured to move linearly along the first support surface to enable the carriage and the motor to selectively translate within the handle, and
      a second support surface coupled to the handle and supporting the second bearing,
   wherein the second bearing is configured to move linearly along the second support surface to enable the carriage and the motor to selectively translate within the handle.
19. The tissue-removing catheter according to paragraph 18, wherein the first support surface is an interior surface of a rail defining an elongate interior space extending along a length of the rail, wherein the first bearing includes a rail roller bearing received in the interior space and engaging the interior surface.
20. The tissue-removing catheter according to paragraph 19, wherein the second support surface is an internal shoulder spaced apart from and generally parallel to the rail, wherein the second bearing includes a shoulder roller bearing engaging the internal shoulder.

When introducing elements of the present invention or the one or more embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

As various changes could be made in the above apparatuses, systems, and methods without departing from the scope of the invention, it is intended that all matter contained in the above description and shown in the accompanying drawings shall be interpreted as illustrative and not in a limiting sense.

1-15. (canceled)

16. A tissue-removing catheter for removing tissue in a body lumen, tissue-removing catheter comprising:
an elongate body having an axis and proximal and distal end portions spaced apart from one another along the axis, the elongate body being sized and shaped to be received in the body lumen;
a handle at the proximal end portion of the elongate body;
a motor in the handle, the motor being operatively coupled to the elongate body to drive rotation of the elongate body about the axis of the elongate body;
a tissue-removing element mounted on the distal end portion of the elongate body, the tissue-removing element being configured to remove the tissue as the tissue-removing element is rotated by the elongate body within the body lumen; and
a slide assembly at least partially received in the handle, the slide assembly including
a carriage coupled to and supporting the motor,
at least one roller bearing coupled to the carriage, and
a track coupled to the handle and supporting the at least one roller bearing, wherein the at least one roller bearing is configured to move linearly along the track to enable the carriage and the motor to selectively translate within the handle.

17. The tissue-removing catheter of claim 16, wherein the track includes a rail having an interior surface defining an interior space extending along a length of the rail, wherein the at least one roller bearing includes a rail roller bearing rotatably engaging the interior surface within the interior space.

18. The tissue-removing catheter of claim 17, wherein the interior surface of the rail includes opposing surface portions and a cross-wise surface portion extending between the opposing surface portions together defining a channel of the interior space, wherein the rail roller bearing is captured within the channel of the interior space.

19. The tissue-removing catheter of claim 18, wherein the rail roller bearing rotatably engages the opposing surface portions of the interior surface of the rail defining the channel.

20. The tissue-removing catheter of claim 17, wherein the rail roller bearing includes
a first rail roller rotatable within a first rail roller plane, and
a second rail roller rotatable within a second rail roller plane that is generally transverse to the first rail roller plane.

21. The tissue-removing catheter of claim 20, wherein the first rail roller includes a plurality of first rail rollers spaced apart from one another along length of the carriage.

22. The tissue-removing catheter of claim 21, wherein the second rail roller includes a plurality of second rail rollers spaced apart from one another along the length of the carriage.

23. The tissue-removing catheter of claim 21, wherein the second rail roller is disposed longitudinally between adjacent ones of the plurality of first rail rollers relative to the length of the carriage.

24. The tissue-removing catheter of claim 17, wherein the interior surface of the rail includes opposing surface portions extending between the opposing surface portions together defining a slot, wherein the rail roller bearing is disposed within the slot.

25. The tissue-removing catheter of claim 17, wherein the track further includes an internal shoulder spaced apart from and generally parallel to the length of the rail, wherein the at least one roller bearing further includes a shoulder roller bearing rotatably engaging the internal shoulder.

26. The tissue-removing catheter of claim 16, wherein the track includes an internal shoulder extending inward relative to the handle.

27. A tissue-removing catheter for removing tissue in a body lumen, tissue-removing catheter comprising:
an elongate body having an axis and proximal and distal end portions spaced apart from one another along the axis, the elongate body being sized and shaped to be received in the body lumen;
a handle at the proximal end portion of the elongate body;
a motor in the handle, the motor being operatively coupled to the elongate body to drive rotation of the elongate body about the axis of the elongate body;
a tissue-removing element mounted on the distal end portion of the elongate body, the tissue-removing element being configured to remove the tissue as the tissue-removing element is rotated by the elongate body within the body lumen; and
a slide assembly in the handle, the slide assembly including
a carriage coupled to and supporting the motor,
at least one bearing coupled to the carriage, the bearing including a first rail bearing moveable within a first rail plane, and a second rail bearing moveable within a second rail plane that is generally transverse to the first rail plane, and
a track coupled to the handle and supporting the at least one bearing of the slide assembly, wherein the at least one bearing is configured to move linearly along the track to enable the carriage and the motor to selectively translate within the handle, the track including a rail having an interior surface defining an interior space extending along a length of the rail, wherein at least one of the first rail bearing and the second rail bearing engages the interior surface within the interior space.

28. The tissue-removing catheter of claim 27, wherein the first rail bearing includes a plurality of first rail bearings spaced apart from one another along a length of the carriage.

29. The tissue-removing catheter of claim 28, wherein the second rail bearing includes a plurality of second rail bearings spaced apart from one another along the length of the carriage.

30. The tissue-removing catheter of claim 28, wherein the second rail bearing is disposed longitudinally between adjacent ones of the plurality of first rail bearings relative to the length of the carriage.

31. The tissue-removing catheter of claim 27, wherein the second rail plane is generally orthogonal to the first rail plane.

32. The tissue-removing catheter of claim 27, wherein the track further includes an internal shoulder spaced apart from and generally parallel to a portion of the rail, wherein the at least one bearing further includes a shoulder bearing engaging the internal shoulder.

33. A tissue-removing catheter for removing tissue in a body lumen, tissue-removing catheter comprising:
an elongate body having an axis and proximal and distal end portions spaced apart from one another along the axis, the elongate body being sized and shaped to be received in the body lumen;
a handle at the proximal end portion of the elongate body;
a motor in the handle, the motor being operatively coupled to the elongate body to drive rotation of the elongate body about the axis of the elongate body;
a tissue-removing element mounted on the distal end portion of the elongate body, the tissue-removing element being configured to remove the tissue as the tissue-removing element is rotated by the elongate body within the body lumen; and
a slide assembly in the handle, the slide assembly including
a carriage coupled to and supporting the motor,
at least one bearing coupled to the carriage, the bearing including a first rail bearing moveable within a first rail plane, and a second rail bearing moveable within a second rail plane angularly offset to the first rail plane,
a first track coupled to the handle and supporting the at least one bearing, wherein the at least one bearing is configured to move linearly along the first track to enable the carriage and the motor to selectively translate within the handle,
a second track coupled to the handle and supporting the at least one bearing, wherein the at least one bearing is configured to move linearly along the first track to enable the carriage and the motor to selectively translate within the handle.

34. The tissue-removing catheter of claim 33, wherein the first track includes a rail having an interior surface defining an elongate interior space extending along a length of the rail, wherein the at least one bearing includes a rail bearing received in the interior space and moveably engaging the interior surface.

35. The tissue-removing catheter of claim 33, wherein the second track includes an internal shoulder spaced apart from and generally parallel to at least one rail, wherein the at least one bearing includes a shoulder bearing moveably engaging the internal shoulder.
